(19) United States
(12) Patent Application Publication
(10) Pub. No.: US 2007/0208163 A1
(43) Pub. Date: Sep. 6, 2007

(54) Method for treatment of protein precipitates

(75) Inventors: Ole Elvang Jensen, Vanlose (DK); Are Bognes, Niva (DK); Martijn Nico Gerard Marie Wiertz, Bagsvaerd (DK); Arne Staby, Bagsvaerd (DK); Marc Antonius Theodorus Bisschops, Breda (NL); Tiemens Geert Peter Reijns, The Hague (NL)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/327,953

(22) Filed: Jan. 9, 2006

Related U.S. Application Data
(63) Continuation of application No. PCT/DK04/00437, filed on Jun. 21, 2004.
(60) Provisional application No. 60/501,164.
(60) Provisional application No. 60/505,183, filed on Sep. 23, 2003.

(30) Foreign Application Priority Data
Jul. 10, 2003 (DK) PA 2003 01050

(51) Int. Cl. A61K 38/28; C07K 14/605

(52) U.S. Cl. 530/303; 530/412; 530/399

(57) ABSTRACT

The present invention relates to a method of washing and concentrating of protein precipitates by use of centrifugal forces. The proteins may be insulin, insulin analogues or insulin derivatives or GLP-1 or GLP-2 and analogues and derivates thereof such as acylated proteins.

### CROSS-REFERENCE TO RELATED APPLICATIONS

This application is a continuation of International Application No. PCT/DK2004/000437, filed Jun. 21, 2004, which claims priority from Danish Patent Application No. PA 2003 01050 filed Jul. 10, 2003 and to U.S. Patent Application Nos. 60/501,164 filed Sep. 8, 2003; 60/505,183 filed Sep. 23, 2003.

### FIELD OF THE INVENTION

The present invention relates to a method for washing and concentrating of protein precipitates by use of centrifugal forces.

### BACKGROUND OF THE INVENTION

Most proteins for pharmaceutical use are today made by recombinant production technology which encompasses fermentation in large scale of transformed cell lines or microorganisms comprising inserted DNA capable of expressing and optionally secreting the desired protein. The directly expressed product may either be the ultimate product or may be a precursor which by further in vitro steps can be converted into the final product. Such in vitro steps may be enzymatic or chemical cleavage if the precursor is a fusion product comprising peptide sequences which are not wanted in the final products. The in vitro conversion may also comprise one or more chemical conversions, e.g. an acylation step where an acyl group is introduced in one or more positions of the protein molecule to introduce altered biological behavior of the protein, e.g. a more protracted mode of action.

Irrespective of whether the protein is the directly expressed product or is further converted by intro steps, the product is subjected to multiple purification and concentration steps such as precipitation, centrifugation, filtration, crystallization and chromatographic procedures in order to remove all impurities and by-products originating from the production method.

Traditionally, concentration, washing and solvent exchange of protein precipitates or crystal suspensions are performed by repetitive steps of precipitation, centrifugation, isolation and resuspension or for some crystals with suitable microstructure, by filtration and washing on the filter. Methods for washing and separating of precipitates are disclosed in U.S. Pat. Nos. 4,436,631; 6,180,394; 4,350,283; 3,825,175; 4,798,579 and 4,670,002.

U.S. Pat. No. 6,180,394 discloses a method for chromatographic purification in a fluidised bed. A product is bound to a resin and washed with a suitable solvent. Then the product is eluted with another solvent. The process is operated in a centrifugal force field to accelerate the sedimentation.

U.S. Pat. No. 4,350,283 and U.S. Pat. No. 4,670,002 both disclose a method for separating particles according to size in a centrifugal force field. The heavy fraction accumulates inside the separation chamber, while the lighter fraction is discharged with the fluid at the other end of the separation chamber.

The hereto used methods for separating of protein precipitates normally require use of a fairly large numbers of repetitive steps, such as washing and resuspending of the precipitate, and are labor intensive and require large solvent volumes for washing and solvent exchange. These methods also induce particle aggregation, resulting in change of the particle properties and undesired the inclusion of solvent and solutes. Furthermore, manual handling of precipitates in open systems when emptying filters, centrifuges and the like may increase risk of product contamination. Finally, the risk of yield losses is increased for each additional step reducing the economy of the overall process.

Thus, there is a need in the art for a more efficient and less labor intensive process for large scale operation in purifying and concentration of proteins.

### SUMMARY OF THE INVENTION

The present invention is related to a method for washing and optionally concentrating a protein precipitate comprising
a) feeding a suspension of a protein precipitate in a first solvent to a rotating chamber and simultaneously discharging solvent without precipitate while establishing a fluidized zone;
b) introducing a second solvent into the rotating chamber and simultaneously discharging solvent without precipitate, whereby the first solvent is partly or completely exchanged by the second solvent while maintaining a fluidized zone; and
c) collecting the protein.

It is important for the efficiency of the present process that the fluidized zone established in step a) is maintained in step b).

The fluidized zone is established by adjusting feeding rate and rotating speed of the rotating chamber as will be evident to the expert in the art.

In one embodiment of the present method, the feeding of the suspension of the protein in the first solvent and the feeding of the second solvent to the rotating chamber is made through an inlet at the outer peripheral zone of the rotation chamber whereas the discharge of solvent is made at an outlet at the inner peripheral zone of the rotation chamber.

During the feeding of the suspension of the protein precipitate in step a) the centrifugal forces will move the suspended particles towards the outer peripheral zone of the rotating chamber. When feeding of the suspension is continued under simultaneously discharging of the solvent at the same rate, the concentration of the protein precipitate in the first solvent will continuously increase because only solvent but no particles are discharged from the system. Step a) is continued until the desired concentration of the suspended protein particles is obtained in the rotating chamber at a given combination of rotation speed and feed rate. An obvious upper limit for the degree of concentration is reached when suspended protein particles are seen in the outlet.

In a further embodiment the feeding in step a) is terminated before introduction of the second solvent in step b). In this embodiment the process is run as a batch process and the same inlet and outlet may be used for feeding of the first and second solvent and for discharging solvent from the rotating chamber, respectively.

Feeding of the second solvent in step b) to the rotating chamber may conveniently take place through the same inlet as the first solvent. However, the second solvent may also be introduced through another inlet situated at the peripheral end of the rotating chamber.

During feeding of the second solvent in step b), solvent is withdrawn with the same rate through an outlet which may and may not be the same as the outlet for the first solvent.

The continued operation of the apparatus will now cause an exchange of the first solvent by the second solvent, e.g. exchange of one buffer with another buffer or exchange of a buffer with water or with another suitable solvent.

The concentration of the protein precipitate in the fluidized zone is controlled by the feed rate and the centrifugal force. The concentration of the protein precipitate can be increased by a factor of at least about two compared to the concentration of the protein in the first solvent, preferably by a factor of at least about 3 and more preferably by a factor of at least about 5. In a typically embodiment of the present invention the concentration of the protein can be increased by 50-400% compared to the concentration in the first solvent.

The first and the second solvent will typically have different densities. If the density of the second solvent is different from the density of the first solvent, the density may be gradually changed in discrete steps or may be changed by means of a continuous gradient.

Exchange of solvent is easier if the density of the second solvent is higher than the density of the first solvent. However, use of a second solvent with the same or substantially the same density or with a lower density than the first solvent is also possible. In that case a repetitive washing with the solvent or a wash with solvents with decreasing densities may be advantageous in order to reduce the effect of mixing and turbulence caused by density variation between the feed on one side and the wash buffer or the solvent on the other side. Alternatively, a solvent gradient may be used.

In still a further embodiment the first solvent is replaced with a second solvent with the same or substantially the same viscosity or with a higher or a lower viscosity.

If the viscosity of the second solvent is different from the viscosity of the first solvent it may be gradually changed in discrete steps or it may be changed by means of a continuous gradient.

Collection of the protein in step c) can be done by any convenient means such as manual collection; adjusting the flow rate; adjusting the flow direction; adjusting the speed of rotation, introducing gas into the system or dissolving the protein in a suitable solvent; or any combination thereof.

In one embodiment steps a); b) and c) are carried out consecutively in that order, however, steps a); b) and c) may also be carried out simultaneously in a continuous process. In a further embodiment step a) and b) are carried out simultaneously and in still a further embodiment step b) and c) are carried out simultaneously.

If steps a); b) and c) are carried out simultaneously, the first solvent may be introduced at the center of the chamber or close to the center of the chamber at the inner peripheral zone of the chamber while the supernatant is withdrawn from a separate outlet at the inner peripheral zone of the chamber. The second solvent will in this embodiment be introduced to the chamber counter currently to the centrifugal force field at the outer peripheral end and washed precipitate will be withdrawn in a zone between the inner and outer end of the chamber through a separate outlet.

Feeding rates and rotation speeds may be varied during the operation of the individual steps of the process. Thus they may be decreased or increased to establish or to maintain the fluidized bed or to discharge product from the rotating chamber. Furthermore, the feed rate and rotation speed in the individual steps a), b) and c) of the process may be the same or different.

The process temperature may be controlled by appropriate means and may include direct cooling of the chamber, cooling of the seals, where most of the heat is generated, and/ or cooling of the feed and wash solutions. The temperature may be adjusted within a range from about minus 5° C. to about 100° C.

In one embodiment the process temperature can be independently controlled in step a); b) and c) respectively.

If gas is used to remove the protein precipitate from the rotation chamber the gas may be selected from the group consisting of air, steam and inert gasses, such as nitrogen, or any combination thereof.

In one embodiment the first solvent is exchanged by the second solvent by at least about 75%, about 80%, about 85%, about 90% or about 95% v/v.

The use of a centrifugal force field makes it possible to combine several purification steps such as concentration, washing and solvent exchange in one apparatus in a very efficient operation in large scale and thus saves time and amount of solvent and increases the yield. Furthermore, the process can be conducted in a closed system thereby reducing risk of product contamination and enables concentration and optionally washing of precipitates of proteins which are fragile or susceptible to attrition.

### BRIEF DESCRIPTION OF THE DRAWINGS

The invention is further described in the enclosed drawings wherein

FIG. 1 shows a cross section of a centrifugal contactor used in the examples;

FIG. 2 schematically shows a set up for running the method according to the invention;

FIG. 3 schematically shows step a), b) and c) of the method according to the invention in a batch system;

FIG. 4 schematically shows a continuous operation of the method according to the invention;

FIG. 5 is a graphic presentation of the Dimensionless effluent conductivity curve of the washing of a high-density crystal suspension using a low-density buffer; and

FIG. 6 is a graphic presentation of an effluent conductivity curve of the washing of a high-density crystal suspension using intermediate and low-density buffers

### DETAILED DESCRIPTION

Abbreviations and Nomenclature

Washing: With washing is meant a process wherein the solvent in which the protein precipitate is suspended is partly or completely exchanged or displaced by another solvent. The purpose of the washing method may be to remove salts contained in the first solvent or to substitute one buffer for another buffer.

Protein: The expression protein is intended to include peptides and polypeptides.

Precipitate: The precipitate can be in any physical form including crystalline form, amorphous form and aggregates and any other non dissolved state of the protein.

Fluidized zone: Fluidization occurs when particles are pushed against the centrifugal force in the chamber at a velocity corresponding to their sedimentation velocity.

Displacing: Replacement of one solvent with another with no or very limited convective mixing.

Aqueous solution: With an aqueous solution is meant a solution containing water and other substances, such as salts, buffering component, additives and/or organic components.

The protein may be any protein, e.g. aprotinin, tissue factor pathway inhibitor or other protease inhibitors, insulin or insulin precursors, human or bovine growth hormone, interleukin, glucagon, GLP-1, GLP-2, IGF-I, IGF-II, tissue plasminogen activator, transforming growth factor α or β, platelet-derived growth factor, GRF (growth hormone releasing factor), immunoglubolines, EPO, TPA, protein C, blood coagulation factors such as FVII, FVIII, FIV and FXIII, exendin-3, exentidin-4, and enzymes and functional analogues thereof. In the present context, the term “functional analogue” is meant to indicate a protein with a similar function as the native protein. The protein may be structurally similar to the native protein and may be derived from the native protein by addition of one or more amino acids to either or both the C- and N-terminal end of the native protein, substitution of one or more amino acids at one or a number of different sites in the native amino acid sequence, deletion of one or more amino acids at either or both ends of the native protein or at one or several sites in the amino acid sequence, or insertion of one or more amino acids at one or more sites in the native amino acid sequence. Furthermore the protein may be acylated in one or more positions, vide WO 98/08871 which discloses acylation of GLP-1 and analogues thereof and in WO 98/08872 which discloses acylation of GLP-2 and analogues thereof. An example of an acylated GLP-1 derivative is Lys²⁶(N^{ε}-tetradecanoyl)-GLP-1₍₇₋₃₇₎ which is GLP-1₍₇₋₃₇₎ wherein the ε-amino group of the Lys residue in position 26 has been tetradecanoylated.

An insulin analogue is an insulin molecule having one or more mutations, substitutions, deletions and or additions of the A and/or B amino acid chains relative to the human insulin molecule. The insulin analogues are preferably such wherein one or more of the naturally occurring amino acid residues, preferably one, two, or three of them, have been substituted by another codable amino acid residue. Thus position 28 of the B chain may be modified from the natural Pro residue to one of Asp, Lys, or Iie. In another embodiment Lys at position B29 is modified to Pro; Also, Asn at position A21 may be modified to Ala, Gln, Glu, Gly, His, Ile, Leu, Met, Ser, Thr, Trp, Tyr or Val, in particular to Gly, Ala, Ser, or Thr and preferably to Gly. Furthermore, Asn at position B3 may be modified to Lys. Further examples of insulin analogues are des(B30) human insulin, insulin analogues wherein PheB1 has been deleted; insulin analogues wherein the A-chain and/or the B-chain have an N-terminal extension and insulin analogues wherein the A-chain and/or the B-chain have a C-terminal extension. Thus one or two Arg may be added to position B1.

An example of a precursor is an insulin precursor with an amino acid sequence B(1-29)-Ala-Ala-Lys-A(1-21) wherein A(1-21) is the A chain of human insulin and B(1-29) is the B chain of human insulin in which Thr(B30) is missing. This insulin precursor may be converted in human insulin by enzymatic cleaving off the Ala-Ala-Lys bridge, connecting the amino acid residue in position B29 with the amino acid in position A21, and enzymatic coupling of a Thr amino acid to the B29 amino acid residue. Other insulin precursors may comprise an N-terminal extension to the B-chain which is the later on cleaved of by suitable enzymatic or chemical treatment, see U.S. Pat. No. 6,521,738, WO 97/22706, WO 97/00581 and WO 00/04172.

Other insulin intermediates are such which can be converted into the desired insulin product by e.g. acylation or pegylation to form a protracted insulin molecule. Acylated insulins are disclosed in EP 792290B which discloses acylated insulins and acylated insulin analogues. Examples of acylated insulins are such being acylated in the B29 position of human insulin or desB30 human insulin or in position B28 in a modified human insulin with a Lys in B28 and a Pro in B29, e.g. N^{εB29}-tetradecanoyl des(B30) human insulin; N^{εB29}-(lithocholoyl-γ-Glu) des(B30) human insulin; N^{εB28}-tetradecanoyl Lys^{B28}Pro^{B29} human insulin; N^{εB29}-tetradecanoyl Asp^{B28} human insulin; N^{εB29}-tetradecanoyl Gln^{B3} des(B30) human insulin), N^{εB29}-tridecanoyl human insulin, N^{εB29}-tetradecanoyl human insulin, N^{εB29}-decanoyl human insulin, and N^{εB29}-dodecanoyl human insulin.

The first and second solvent, which may the same or different, may be any suitable solvent dependent on the protein in question such as water, an aqueous solution, an organic solvent, or any mixtures thereof. If the solvents are the same, the effect of the process is mainly to increase the concentration of the precipitate in the solvent, whereas if the solvents are different, the process will enable an up-concentration and a washing of the precipitate.

Thus the first solvent may be an aqueous acetate solution containing a suitable salt whereas the second solvent may be the same acetate solution but without salt. The first solvent may also be a buffer with one pH whereas the second solvent may be a different buffer with another pH and optionally containing a salt. The first solvent may furthermore be a mixture of water and an organic solvent whereas the second solvent may be one of these ingredients, i.e. either water or organic solvent.

In one embodiment of the present invention the salt component is selected from the group consisting of organic or inorganic salts and mixtures thereof, preferably NaCl, KCl, NH₄Cl, CaCl₂, sodium acetate, potassium acetate, ammonium acetate, sodium citrate, potassium citrate, ammonium citrate, sodium sulphate, potassium sulphate, ammonium sulphate, calcium acetate or mixtures thereof.

The term “a buffer” as used herein, is intended to include any buffer including but not limited to: citrate buffers, phosphate buffers, tris buffers, borate buffers, lactate buffers, glycyl glycin buffers, arginine buffers, carbonate buffers, acetate buffers, glutamate buffers, ammonium buffers, glycin buffers, alkylamine buffers, aminoethyl alcohol buffers, ethylenediamine buffers, tri-ethanol amine, imidazole buffers, pyridine buffers and barbiturate buffers and mixtures thereof (cf. Remington's Pharmaceutical Sciences, Gennaro, ed., Mack Publishing Co., Easton, Pa., 1990, or Remington: The Science and Practice of Pharmacy, 19th Edition (1995), or handbooks from Amersham Biosciences). Publishing Co., Easton, Pa., 1990, or Remington: The Science and Practice of Pharmacy, 19th Edition (1995), or handbooks from Amersham Biosciences).

The present invention will be described in further details below with reference to the figures and the examples.

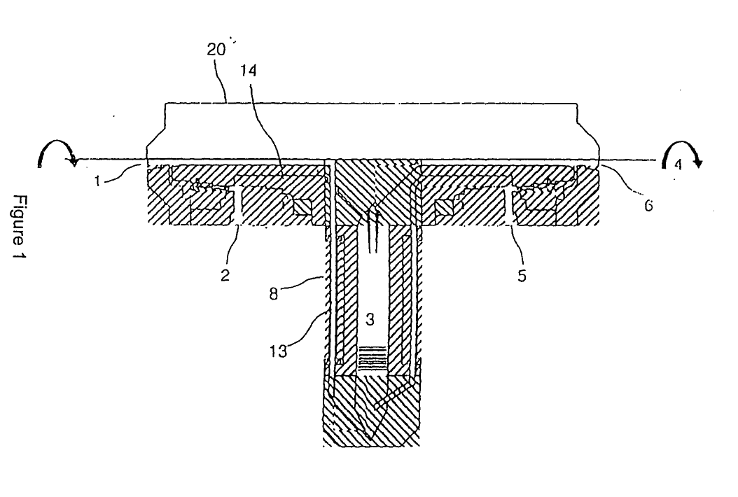

FIG. 1 illustrates a centrifugal contactor (20) used in the examples. It consists of two static cylinders (8) each containing an inlet (1) and an outlet (2) connected to a rotating chamber (3) via tubes (13) and (14). In a continuous operation of the method further ports (5) and (6) are used. In a stepwise or batch operation preferably only one inlet (1) and one outlet (2) are used.

The centrifugal contactor can rotate around a fixed rotor shaft (4). The speed of rotation of the device can be between 1 and 5000 rpm, but may be as high as 10,000 or 20,000 rpm. The correlation between G forces and rpm is as follows: G-force=rpmˆ2*r*(2π/60)ˆ2*(1/g), where: rpm=rotations per minute, r=radius of the centrifuge and g=gravity constant (9.8 m/sˆ2).

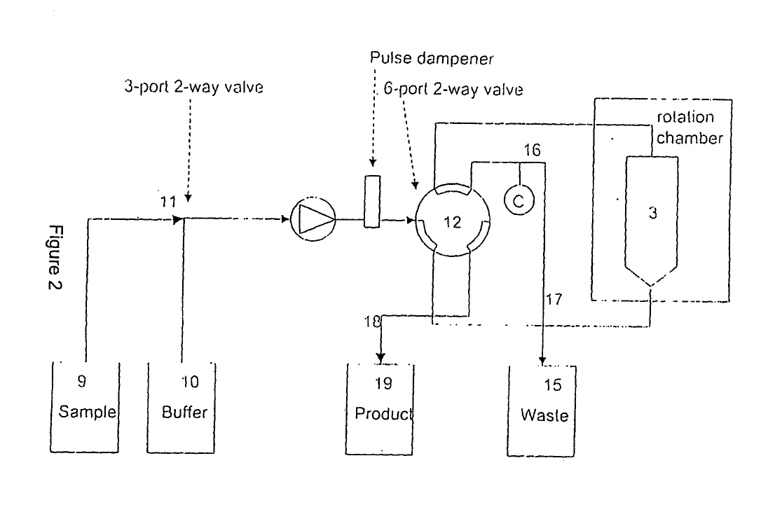

FIG. 2 describes a general overview of the method according to the present invention. In FIG. 2 (9) is a storage container containing the feed suspension. (10) is a storage container with buffer used to wash the protein precipitate and (11) is a 3-port 2-way valve. By switching the 3-port 2-way valve, the liquid entering the centrifugal contactor (3) can be selected. The flow direction can be determined by switching a 6-port 2-way valve (12). In an effluent tube (17) connected to a waste container (15) the liquid passes a conductivity probe (16) (type Consort C-832). Concentrated and washed product is collected through an outlet (18) and collected in a container (19).

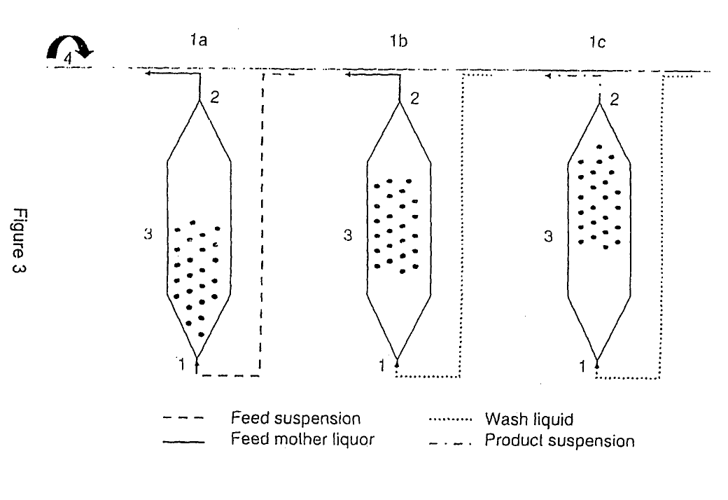

FIG. 3 describes an embodiment of the present invention wherein step a), b) and c) are conducted stepwise, i.e. the loading, washing and recovery of the protein particles are conducted in separate steps in the rotation chamber (3). The feed suspension is the protein precipitate suspended in the first solvent and the wash liquid is the second solvent. Feed mother liquor is solvent (supernatant) without or substantially without protein precipitate.

In step a) (1a in FIG. 3) the particles are fed to the rotating chamber (3) from inlet (1) at the end of the rotating chamber (3) furthest form the rotor shaft (4), the outer peripheral end of the rotating chamber. Excess of the first solvent, the feed mother liquor, is discharged from the rotation chamber (3) through outlet (2) close to the rotation shaft (4). As the protein particles are forced in an outward motion due to the centrifugal force while liquid is fed in the opposite direction, a fluidized bed of suspended particles is created in the rotating chamber. Therefore, the loading volume of feed suspension is not limited to the volume of the rotating chamber (3) and more than one chamber volume can be fed to the contactor while at the same time increasing the concentration of the protein particles in the suspension in the fluidized zone and discharging depleted first solvent (feed mother liquor) through outlet (2). When the feed suspension has been loaded to the chamber to the desired extent, feeding of the feed suspension is terminated.

In step b) (1b in FIG. 3) new fresh wash liquid, the second solvent, is fed to the chamber through inlet (1). Hereby the first solvent is displaced with the wash liquid (the second solvent), while maintaining the fluidized zone of particles in the rotation chamber (3). Excess of solvent is at the same time discharged through outlet (2).

In step c) (1c in FIG. 3) the washed and concentrated particles can now be recovered in fresh wash liquid through outlet (2), by reducing the centrifugal force or increasing the wash flow rate. Alternatively the concentrated particles may be recovered through inlet (1) by reversing the wash flow direction.

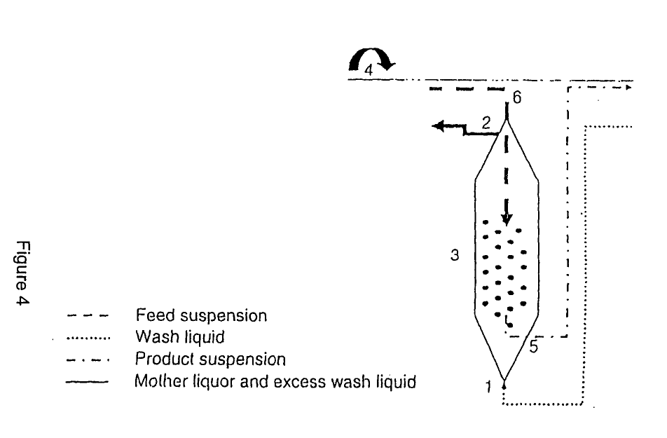

FIG. 4 describes loading, washing and recovery of protein particles in the rotating chamber (3) in a continuous operation. The first step of this embodiment of the present invention is identical to step 1a in FIG. 3. In the next step of the process, feed suspension is continuously fed through port (6) close to the middle of the chamber (3) and wash liquid is continuously fed through inlet (1) to the fluidized bed, with a flow rate large enough to displace the feed mother liquor from at least that part of the fluidized bed where the washed product suspension is recovered through port (5). During further processing, the remaining feed mother liquor and possibly part of the washing liquid is continuously discarded through outlet (2).

The present method is in particular useful to treat sticky precipitates and crystals which may be more difficult to treat in a conventional batch filtration or centrifugation step in large scale.

### EXAMPLES

In the following examples dry insulin crystals were used as a model. However, the present method is suitable for a vide variety of proteins as will be obvious to the person skilled in the art. The dry insulin crystals contained 82.7% w/w insulin. The feed suspension for the experiments was prepared by suspending dry insulin crystals in a first solvent.

The “first solvent” in Examples 1, 2 and 3 consisted of am aqueous solution containing:
500 mM Sodium dihydrogen phosphate (NaH₂PO₄)
10 mM Sodium acetate (NaH₃CCOO)
13 mM Sodium chloride (NaCl)
3.7 mM Hydrochloric acid (HCl)
adjusted to pH 4.8-4.9
Conductivity: 28.5 mS/cm
Density: 1076.2 g/l
   Another buffer used in the examples, the “second solvent” consisted of an aqueous solution containing:
10 mM Sodium acetate (NaH₃CCOO)
13 mM Sodium chloride (NaCl)
3.7 mM Hydrochloric acid (HCl)
adjusted to pH 4.8-4.9
Conductivity: 2.4 mS/cm
Density: 998.2 g/l

The solvent densities were determined in a DMA-48 density meter (Anton-Paar GmbH, Graz, Austria). The density of the first solvent was determined to be 1076.2 g/l, the density of the second solvent was determined to be 998.2 g/l, and a 100 g/l suspension of dry insulin crystals in the first solvent was determined to have a density of 1428 g/l. The pH and conductivity were determined with a C-832 pH/CfT meter (Consort, Turnhout, Belgium). Insulin concentrations were measured retrospectively using UV absorbance.

The following non-limiting examples illustrate the invention. Examples 1, 2 and 3 all use an insulin crystal suspension as the feed. Example 4 describes the application of the invention to an insulin precipitate suspension.

The equipment used is a centrifugal contactor with a chamber volume of 65 ml (1 chamber volume) and a dead system volume of 104 ml (1.6 chamber volume). Only one of the two chambers was used for conducting the washing method according to the present invention.

### Example 1

Concentration of an Insulin Crystal Suspension

A crystal suspension was treated in the claim method in a centrifugal contactor shown in FIG. 1.

Solvents could enter the rotation chamber at the top and the bottom which are both connected to the static in- and outlets (1) and (2) via a rotating seal and connected to the rotating part of the device. The rotation chamber was 2.5 cm in diameter and had a volume of approximately 65 ml. The chamber contained no internals (such as a disc stack, baffles or distributors) except for a screen at the bottom at inlet (1). The speed of rotation was between 0 and 2500 rpm.

The feed material used in this example was a 24 g/l insulin crystal suspension in the second solvent, which was prepared by adding dry insulin crystals to this solvent. Its exact insulin concentration was determined by dissolving the solids and determining the total protein content using a spectrophotometric method.

The centrifugal contactor was filled with buffer free of insulin before the start of the actual experiment. The contactor was then operated at 1500 rpm and a total of 919.4 g of insulin crystal suspension was pumped into the contactor at a rate of 35.8 g/min. At the end of this loading stage, the feed pump was switched to buffer at the same flow rate and in the same flow direction. After about 1.5 minutes, the speed of rotation of the contactor was instantaneously brought down to 200 rpm and the flow direction was reversed. The buffer was now fed to the contactor via inlet 2. Several fractions of the product of this step were collected; the most concentrated of these fractions had a concentration of 76.8 g/l insulin.

### Example 2

Single-Stage Washing of an Insulin Crystal Suspension

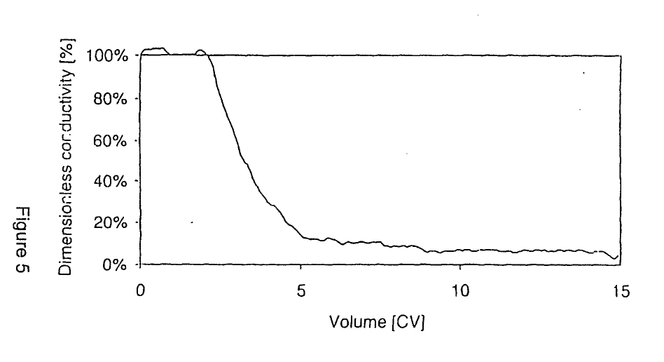

A total of 922.4 g of an insulin crystal suspension containing 18.7 g/l insulin in the first solvent was loaded into the same centrifugal contactor as used in Example 1 at a rate of 37.1 g/min. The contactor was operated at 1500 rpm. After this loading step, the suspension inside the rotating chamber was washed using the second solvent at a flow rate of 37.4 g/min for a total of 48 min. The conductivity of the contactor effluent was monitored during the wash step. In this situation the conductivity of the effluent can be used to determine which solvent that is discarded from the system. The dimensionless results (normalized to the conductivity: conductivity of the second solvent=0%, conductivity of the first solvent is 100%) are shown in FIG. 5.

The dead volume of the system (total volume including inlet pipes and the like) was determined to be 1.6 chamber volume. Using this figure, the volume of wash buffer necessary for reaching a certain washing efficiency can be read from the graph in FIG. 5:

1.7 chamber volumes for 50% efficiency

5.6 chamber volumes for 90% efficiency

13.0 chamber volumes for 95% efficiency

After the wash step, the flow direction was reversed and the speed of rotation of the contactor was brought down instantaneously to 200 rpm. This resulted in a number of product fractions, the most concentrated of which had a total mass of 76 g and an insulin concentration of 33 g/l.

### Example 3

Two-Stage Washing of an Insulin Crystal Suspension

A total of 980.8 g of an insulin crystal suspension containing 22.5 g/l insulin in the first solvent was loaded into the same centrifugal contactor as used in Example 1 at a rate of 39.9 g/min. The contactor was operated at 1500 rpm. After this loading step, the suspension inside the rotating chamber was washed in two steps at a flow rate of 36.8 g/min. In the first step a total of 146.8 g of a buffer containing 50% first solvent and 50% second solvent was used. In the second step a total of 150.6 g of the second solvent was used.

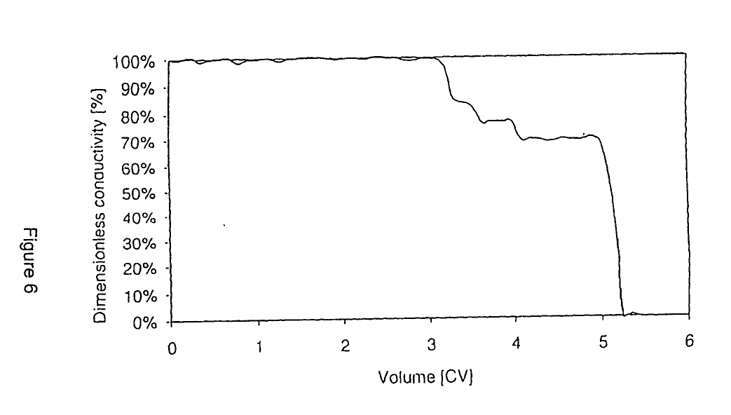

The conductivity of the contactor effluent was monitored during the wash steps and the results are shown in FIG. 6 which shows the effluent conductivity curve of the washing of a high-density crystal suspension using intermediate and low-density buffers.

As in example 1 the normalized conductivity is used as a reference for the effluent composition. 0% corresponding to 2.4 mS/cm corresponds to pure second solvent and 100% corresponding to 28.5 mS/cm corresponds to pure first solvent.

The product suspension conductivity shows that washing using a total of 3.9 chamber volumes of buffer (corrected for the 1.6 CV dead volume of the system) yielded an 89% washing efficiency. In example 2, this efficiency was reached after some 5.6 column volumes.

After the wash steps, the flow direction was reversed and the speed of rotation of the contactor was reduced instantaneously to 200 rpm. This resulted in a couple of product fractions, the most concentrated of which had a total mass of 83.6 g and an insulin concentration of 57.9 g/l.

### Example 4

Concentration of an Insulin Precipitate Suspension

The feed material used in this example was a 1.9 g/l insulin precipitate suspension in a buffer of pH 5.2 containing approximately 18% w/w ethanol and 10 mM NaAc. The solvent density was determined to be 969.9 g/l in a DMA-48 density meter (Anton-Paar GmbH, Graz, Austria). Its exact insulin concentration was measured by centrifuging a sample of the suspension, removing the supernatant, dissolving the solids in a solution similar to the one the precipitates were originally suspended in but at a pH of 3.0 and determining the total protein content using a spectrophotometric method.

A total of 893.9 g of this insulin precipitate suspension was loaded into a centrifugal contactor described in Example 1 at a rate of 5.73 g/min. The contactor was operated at 500 rpm. After this loading stage, the suspension in the chamber was washed using the pH 5.2 buffer at a flow rate of 5.65 g/min for 20 min, after which the flow direction was reversed and the speed of rotation of the contactor decreased instantaneously to 200 rpm. This resulted in a number of product fractions, the most concentrated of which had a total mass of 41.2 g and an insulin concentration of 8.3 g/l.

1. A method for washing and optionally concentrating a protein precipitate, the method comprising
a) feeding a suspension of said protein precipitate in a first solvent to a rotating chamber and simultaneously discharging the first solvent while establishing a fluidized zone;
b) introducing a second solvent into the chamber and simultaneously discharging solvent whereby the first solvent is partly or completely exchanged by the second solvent while maintaining the fluidized zone, and
c) collecting the protein.

2. A method according to claim 1, wherein the feeding of solvent takes place at the outer peripheral portion of the rotating chamber and discharge of solvent takes place at the inner peripheral zone of the rotating chamber.

3. A method according to claim 1, wherein the feeding of the suspension in step a) is terminated before introduction of the second solvent in step b).

4. A method according to claim 1, wherein the concentration of the protein precipitate in the fluidized zone is controlled by the feed rate and the centrifugal force.

5. A method according to claim 4, wherein the concentration of the protein precipitate is increased by a factor of at least about two compared to the concentration of the protein in the feed suspension.

6. A method according to claim 5, wherein the concentration of the protein precipitate is increased by a factor of at least about 3 compared to the concentration of the protein in the feed suspension.

7. A method according to claim 5, wherein the concentration of the protein precipitate is increased by a factor of at least about 5 compared to the concentration of the protein in the feed suspension.

8. A method according to claim 1, wherein the first solvent is replaced with a second solvent with the same or substantially the same density.

9. A method according to claim 1, wherein the first solvent is replaced with a second solvent with a higher density.

10. A method according to claim 1, wherein the first solvent is replaced with a second solvent with a lower density.

11. A method according to claim 9, wherein the density of the second solvent is gradually changed in discrete steps or by means of a continuous gradient.

12. A method according to claim 10, wherein the density of the second solvent is gradually changed in discrete steps or by means of a continuous gradient.

13. A method according to claim 1, wherein the first solvent is replaced with a second solvent with the same or substantially the same or with a higher viscosity.

14. A method according to claim 1, wherein the first solvent is replaced with a second solvent with a higher viscosity.

15. A method according to claim 1, wherein the first solvent is replaced with a second solvent with a lower viscosity.

16. A method according to claim 14, wherein the viscosity of the second solvent is gradually changed in discrete steps or by means of a continuous gradient.

17. A method according to claim 15, wherein the viscosity of the second solvent is gradually changed in discrete steps or by means of a continuous gradient.

18. A method according to claim 1, wherein the first solvent is water, an aqueous solution, an organic solvent, or any mixtures thereof.

19. A method according to claim 1, wherein the first and second solvent are salt containing aqueous buffers.

20. A method according to claim 1, wherein the protein in step c) is collected by one of the following means: manual means; adjusting the flow rate; adjusting the flow direction; adjusting the speed of rotation, introducing gas into the system or dissolving the protein in a suitable solvent; or any combination thereof.

21. A method according to claim 1, wherein steps a); b) and c) are carried out consecutively in that order.

22. A method according to claim 1, wherein steps a); b) and c) are carried out simultaneously.

23. A method according to claim 1, wherein step a) and b) are carried out simultaneously.

24. A method according to claim 1, wherein step b) and c) are carried out simultaneously.

25. A method according to claim 1, wherein the protein precipitate is fragile or susceptible to attrition.

26. A method according to claim 1, wherein the process temperature in the rotating chamber can be controlled within a range from about minus 5° C. to about 100° C.

27. A method according to claim 26 wherein the process temperature can be independently controlled in step a); b) and c) respectively.

28. A method according to claim 20, wherein the gas is selected from the group consisting of air, steam and inert gasses or any combination thereof.

29. A method according to 1, wherein the first solvent is exchanged by the second solvent by at least 75% v/v.

30. A method according to claim 1, wherein the protein is selected from the group consisting of insulin, insulin analogues, acylated insulins, acylated insulin analogues, GLP-1, GLP-1 analogues, acylated GLP-1, acylated GLP-1 analogues, GLP-2, GLP-2 analogues, acylated GLP-2 and acylated GLP-2 analogues.
